(19) United States
(12) Patent Application Publication
(10) Pub. No.: US 2022/0136818 A1
(43) Pub. Date: May 5, 2022

(54) OPTICAL COHERENCE TOMOGRAPHY ANALYSIS METHOD AND APPARATUS

(71) Applicant: CENTRO INTERNAZIONALE DELLA FOTONICA PER ENERGIA (CIFE) IN FORMA ABBREVIATA "CIFE", Milano (IT)

(72) Inventors: Aldo RIGHETTI, MILANO (IT); Maria Chiara UBALDI, MILANO (IT); Giorgio GRASSO, MONZA (IT)

(73) Assignee: CENTRO INTERNAZIONALE DELLA FORONICA PER ENERGIA (CIFE) IN FORMAL ABBREVIATA "CIFE", MILANO (IT)

(21) Appl. No.: 17/599,975

(22) PCT Filed: Apr. 3, 2020
(86) PCT No.: PCT/EP2020/059501

§ 371 Date: Sep. 29, 2021

(30) Foreign Application Priority Data
Apr. 5, 2019 (IT) 102019000005258

(51) Int. Cl. G01B 9/02091; G01B 9/02004; A61B 3/10

(52) U.S. Cl. CPC G01B 9/02091; A61B 3/102; G01B 9/02004

(57) ABSTRACT

The present invention relates to an optical coherence tomography analysis method, comprising:
Providing a Swept Source Optical Coherence Tomography system (SS-OCT), the SS-OCT system including:
   a light source, tunable over a spectral band, that generates a coherent light signal;
   an optical interferometer for dividing the coherent light signal into a reference arm leading to a reference reflector and a sample arm leading to a sample;
   an optical element to selectively direct a sample light signal exiting the sample arm to a specific portion of the sample, so that for each selection in the optical element a different specific portion of the sample is illuminated;
   an optical detector for detecting an interference signal generated by a combination of reference and sample returning signals from the reference arm and from the sample arm, reflected by the reference reflector and the sample, respectively;
Wherein, for the same selection operated at the optical element level illuminating a specific portion of the sample, the method further comprises:
   sweeping the light source for a time interval ΔT, so that a wavelength of the coherent light signal, leading to the sample light signal illuminating the specific portion of the sample, changes from a minimum wavelength to a maximum wavelength and wherein the wavelength of the coherent light signal reaches the same value between the minimum wavelength to the maximum wavelength at least twice during the sweeping;
   detecting the interference signal generated by the sweeping, including the interference signal generated by the sample returning signals of the at least two coherent light signals having the same wavelength;
   elaborating the detected interference signal generated by the sweeping, including the detected interference signal generated by the sample returning signals of the at least two coherent light signals having the same wavelength, for obtaining an OCT image of the specific portion of the sample.

### TECHNICAL FIELD

The present invention relates to an imagining technique and system for optical coherence tomography (OCT) that uses coherent light to capture two and three dimensional images of samples, in particular when a non-destructive testing of the sample is needed, such as in medical tissues.

### TECHNOLOGICAL BACKGROUND

The functional principle behind OCT imaging is light interference. In an OCT system, the light beam from a source, for example a laser source, is split into two paths by a beam splitter, for example a coupler, directing the split light along two different arms of an interferometer. One arm is generally named reference arm, while the other is named the sample arm. When the light exits the end of either arms, it is shaped by various optical components (mirror, lenses, etc.) to control specific beam parameters such as shape, depth of focus and light intensity distribution. In the reference arm, the light is back reflected by a reference mirror (or any other reflecting surface) and it returns into the interference system, propagating along the same path it came from but in the opposite direction. The same process happens with the light in the sample arm, though in this case the light exiting the arm is backscattered by the sample. In an inhomogeneous sample, different structures within the sample will have different indices of refraction and light will be backscattered when it encounters an interface between materials of different refractive index. The returning lights from both arms recombine, for example at a coupler, and generate an interference pattern, which is recorded by a detector.

It is to be understood that in the present application the term “light” is used in the general sense of “electromagnetic radiation” and it is not limited to radiation in the visible range.

The sample can be any object and the direction of propagation of the light illuminating the sample defines the direction of “depth” of the sample, or Z, while a plane perpendicular to it defines a (X,Y) plane. The scope of OCT is, by means of a (X,Y) scan, to acquire information on the depth of the sample, i.e. information on the sample in the Z direction, which is the direction of propagation of the light beam emitted from the source.

For a particular position of the reference mirror, the light propagating in the reference arm travels a certain optical distance and forms the corresponding interference pattern only with light that has travelled the same optical distance along the sample arm, including the portion of the distance travelled inside the sample. Therefore, when the reference mirror is translated along the propagating direction of light, for different positions of the mirror, the returning reference generates interference patterns with light backscattered from corresponding depths within the sample. In this way, the dependence on depth of backscattered light intensity from beneath the sample surface can be measured.

The OCT signal recorded by the detector during a complete travel of the reference mirror is called a depth scan or A-scan. In order to form an OCT image, the sample beam has to be translated across the sample surface with an A-scan being recorded in each position of the beam. Therefore, a set of consecutive A-scans is obtained from an OCT image or otherwise called B-scan (i.e. set of consecutive A-scans along the X direction). The 3D combination of all A scans and B scans along the Y direction, is called C-scan.

In the scanning above described, there are two mains OCT technologies, time-domain OCT and Fourier domain OCT (also called frequency domain OCT). The latter is further divided in spectral-domain OCT (SD-OCT) and swept-source OCT (SS-OCT). SS-OCT uses a broadband source that scans the sample in a controlled way with a narrow spectral line across the available bandwidth of the source. As a main difference from before, the reference mirror is fixed, i.e. it does not move. The movements of the mirror are “replaced” by the wavelength changes of the light source. As before, however, the reference beam is reflected from the now fixed mirror and forms an interference pattern with the light backscattered by the sample that is subsequently detected by a point detector. Because of the way the source is scanned across the available bandwidth, the output is a wavenumber-dependent photo-current that is recorded by the point detector simultaneously with the scanning of the narrow band source. The quantity of interest, the A-scan, is obtained performing the Fourier transform of the detected signal over one sweep of the source over the available broadband. That is, in SS-OCT, the OCT signal recorded by the detector during a complete sweeping of the source in its bandwidth is called a depth scan or an A-scan. The definition of B and C scans remains unchanged. Since the light from a swept source consists of a source signal with a continuously changing wavelength over time, the coherence length of the scanned laser determines the maximum imaging depth of the system while the wavelength range over which the laser is swept determines the axial resolution of the system. Therefore, a scanning laser with a narrow line width enables a deeper probing depth while a wider sweep range produces OCT images with higher axial resolution.

Due to the fact that SS-OCT systems may also be used to detect images of portions of living bodies, for example of the eye, it is also of importance to generate OCT images in real time, for example to avoid problems related to eye's movements or to be able to perform a 3D imaging of a tissue portion also during a surgical procedure. For example, an OCT image having a scan of 200×200 pixels with a repetition rate of 25 frames per second requires a million sweeping scans per second. In SS-OCT technology, it is therefore important to have a source where generated light beam wavelength can vary as fast as possible.

More in detail, as mentioned, the SS-OCT uses an interferometer. The interference signal obtained, which is a beat signal, has a given frequency. Assuming that the source linearly varies its wavelength (or frequency), then the frequency of the beat signal is determined by the relative delay between the reference signal coming from the reference arm and the signal coming from the sample, thus it depends from the distance between the two surfaces reflecting the two signals exiting the two arms. If Δf is the speed of the source oscillation frequency variation, i.e. the frequency variation rate, its frequency can be written:

$$F = {F}_{0} + ( Δ f ) t$$

where F₀ is the initial frequency and t is the time elapsed from the beginning of the scan. The frequency of oscillation which is detected by the detector of the beat signal (or interference signal) is thus:

$${F}_{b e a t} = F ( t ) - F ( t - T ) = ( Δ f ) T$$

where T is the time delay between the optical signals from the two arms in the interferometer which is in turn equal to

$$T = \frac{z n}{c}$$

where z is the difference in path, c is speed of light and n the refraction index encountered along the light path. The two arms of the interferometer have substantially equal length, thus the length difference z is mainly due to the difference in path caused by the optical signal propagation in the sample.

After the interference signal has been received, it is elaborated, a Fourier transform might for example be performed and the elaborated detected frequencies indicate the depth of the reflecting surfaces of the sample.

If the beat signal from a single reflection related to a specific frequency or wavelength of the optical signal from the source is detected for a time Ts and the source is supposed to have a constant emission power, its Fourier transform can be written as:

$$S ( F ) = \frac{sin ( F * T s )}{F * T s}$$

Thus the depth (or z) resolution of the OCT system depends on the smallest detectable difference between two beat frequencies which, in this case, can be defined as the width of the function sin(x)/x at the first node of the Fourier transform. Without being bound by theory, it results that the depth resolution is equal to

$$Δ Z = \frac{c}{2 n ( T s ) ( Δ f )}$$

where (Δf)(Ts) is the total variation of frequency underwent by the light emitted by the source during a single sweeping. For example, for a spatial z resolution of about 10 μm, the (Δf)(Ts) product (or bandwidth of the source) is about 12,5 THz, which corresponds to about 100 nm.

The single reflection refers to a discontinuity point in the sample that may reflect or diffuse the light and is preferably visualized. It might belong to the surface of the sample. Certain sample might have more than a reflection point for each wavelength, depending on the structure of the sample itself. For example, in case of an eye as sample, for each wavelength and A scan, more than a reflection is generally detected. Each reflection point, belonging to a reflecting surface in a different z position, gives rise to a different beat frequency.

From the calculation above, it is clear that the source to be used in the OCT system needs to be tunable in a wide range, at the same time it also preferably needs to operate in a monomodal regime in the whole required range. Furthermore, the wide tuning has to be performed in a very short time interval to allow the system to be used for example also in the medical field.

Sources used in the SS-OCT systems comprised in the prior art are for example tunable laser sources. These lasers may include an optical gain medium, such as a semiconductor junction, coupled with a cavity having a variable length, such as VCSEL cavity operated by MEMS. Alternatively, a fixed-length cavity can be used, including an optical filter having a tunable band, such as an external cavity laser having an Etalon filter. The sweeping speed depends on the speed of the movable element (in case of MEMS), or the optical filter tuning. Optical movable elements may limit the sweeping speed due to their mechanical inertia and thus generally an optical filter without movable parts is preferred. Optical filters, on the other hand, such as Etalon filters, having such a wide Free Spectral Range (around 100 nm for example) tunable in a very fast time range require the use of ultrafast electro-optic materials such as Lithium niobate, or very special optical crystals. These materials anyhow have small electro-optical coefficients and thus allow small variations of the refractive index.

A possible solution to this problem is for example disclosed in US 2018/013562 where two different sweeping light sources are used in an OCT system, each emitting light at a different bandwidth. The overall needed bandwidth is thus split in two different sources, each of which can have a smaller free spectral range.

### SUMMARY OF THE INVENTION

The present invention relates to a method and a system to perform OCT imaging, and in particular for SS-OCT, in which the used source is tunable in a fast and reliable way and at the same time it provides a bandwidth or free spectral range which is enough for most OCT applications.

As shown in FIG. 1 and from the equations above, it has been shown that in order to have the required depth resolution in a limited amount of time (a quick sweeping time), the variation in wavelength of the light emitted by the source in such short amount of time should be rather broad, i.e. of about or greater than 100 nm. This considerably limits the number of available light sources or requires the use of a very expensive or complex one.

Applicants have noticed that the delay times of the light signals coming from the interferometers have a magnitude of fractions of nanoseconds, while the overall sweeping time for each A-scan is of the order of hundreds of nanoseconds, so there are three orders of magnitude of difference. Furthermore, Applicants have noticed that, for the detected interference signals, positive or negative frequencies difference have the same “effect”. The interference signal, in other words, does not depend on the absolute oscillation frequency, but depends on the (small with respect to the overall sweeping duration) delay between the signals coming back from the sample and the reference and travelling in the two arms of the interferometer, and on the speed in which the frequency (or wavelength) change in time.

Applicants have therefore realized that it is not necessary to increase the wavelength of the light emitted by the laser source continuously during the whole sweeping time. Given a sweeping time ΔT, in which a single A-scan is obtained, the wavelength of the light emitted by the source of the OCT system does not need to increase from a minimum which is obtained at t=0 to a maximum obtained at t=ΔT, as generally assumed in the prior art. The sweeping time ΔT could be divided in sub-intervals, or sub-sweeping times, in each of which the wavelength of the signal emitted by the source might increase or decrease between a minimum and a maximum. This maximum can be smaller, even much smaller, than the maximum wavelength that in a linear regime, i.e. such as in FIG. 1, is to be achieved in order to obtain the desired resolution in depth.

Having a sweeping time in which the source changes its wavelength not only monotonously allows to use in a SS-OCT system light sources which have a more limited wavelength range of variation than what is required in the prior art, without penalizing the time to obtain the overall scan and the image quality (resolution).

According to a first aspect, the invention relates to an optical coherence tomography analysis method, comprising: providing a Swept Source Optical Coherence Tomography system (SS-OCT).

Preferably, the SS-OCT system includes a light source tunable over a spectral band that generates a coherent light signal.

Preferably, the SS-OCT system includes an optical interferometer for dividing the coherent light signal into a reference arm leading to a reference reflector and a sample arm leading to a sample.

Preferably, the SS-OCT system includes an optical element to selectively direct the coherent light signal exiting the sample arm to a specific portion of the sample, so that for each selection in the optical element a different specific portion of the sample is illuminated.

Preferably, the SS-OCT system includes an optical detector for detecting an interference signal generated by a combination of reference and sample returning signals from the reference arm and from the sample arm, reflected by the reference reflector and the sample, respectively.

Preferably, the method, for the same selection in the optical element illuminating a specific portion of the sample, further comprises: sweeping the light source for a time interval ΔT, so that a wavelength of the coherent light signal leading to the sample light signal illuminating the specific portion of the sample changes from a minimum wavelength to a maximum wavelength and wherein the wavelength of the coherent light signal reaches the same value between the minimum wavelength to the maximum wavelength at least twice during the sweeping.

Preferably, the method, for the same selection in the optical element illuminating a specific portion of the sample, further comprises: detecting the interference signal generated by the sweeping, including portions of the interference signal generated by using the sample returning signals of the at least two coherent light signals having the same wavelength.

Preferably, the method, for the same selection in the optical element illuminating a specific portion of the sample, further comprises: elaborating the detected interference signal generated by the sweeping, including portions of the detected interference signal generated by using the sample returning signals of the at least two coherent light signals having the same wavelength, in order to obtain an OCT image of the specific portion of the sample.

According to a second aspect, the invention relates to a Swept Source Optical Coherence Tomography system (SS-OCT).

Preferably, the SS-OCT system includes a light source that generates a coherent light signal that is tuneable over a spectral band.

Preferably, the SS-OCT system includes an optical interferometer for dividing the coherent light signal into a reference arm leading to a reference reflector and a sample arm leading to a sample.

Preferably, the SS-OCT system includes an optical element to selectively direct the coherent light signal exiting the sample arm to a specific portion of the sample, so that for each selection in the optical element a different specific portion of the sample is illuminated.

Preferably, the SS-OCT system includes an optical detector for detecting an interference signal generated by a combination of reference and sample returning signals from the reference arm and from the sample arm, reflected by the reference reflector and the sample, respectively.

Preferably, the SS-OCT system includes a processing unit.

More preferably, the processing unit is programmed for, for the same selection in the optical element illuminating a specific portion of the sample: defining a sweeping time interval ΔT.

Preferably, the processing unit is programmed for, for the same selection in the optical element illuminating a specific portion of the sample: changing the coherent light signal leading to the sample light signal illuminating the specific portion of the sample from a minimum wavelength to a maximum wavelength and in the same sweeping modifying the wavelength of the coherent light signal so that it reaches the same value between the minimum wavelength to the maximum wavelength at least twice during the sweeping.

Preferably, the processing unit is programmed for, for the same selection in the optical element illuminating a specific portion of the sample: elaborating the detected interference signal for obtaining an OCT image of the specific portion of the sample.

The OCT system and method of the invention are used to obtain an OCT scan of a sample. The sample could be a portion of the human body or any other desired element, transparent to the employed wavelength range of the signal emitted by a light source.

In the SS-OCT system of the invention, a coherent light source is used. The light source can emit a coherent light signal having a wavelength which can be varied within a given bandwidth. This light source can be for example a laser, more preferably a tunable laser. The light source, e.g. the tunable laser, has a bandwidth Δλ.

In the SS-OCT system, the coherent light from the coherent light source is split in two by means of an interferometer. The two arms of the interferometers are called sample and reference arms. Thus a portion of the split light signal travels in the sample arm and exits the same, generating the sample light signal. The sample light signal exiting the sample arm illuminates a portion of the sample. In order to select which portion of the sample is to be illuminated to obtain an A-scan of the same, an optical element is provided to select a portion of the sample to illuminate and to move the coherent light coming from the sample arm to different portions of the sample. According to given parameters, the optical element can selectively illuminate with the sample light signal coming from the sample arm a portion of the sample. This illuminated portion changes, i.e. another portion of the sample is selected, when the optical element moves the sample light signal on the sample. The illumination of two different portions of the sample may partially overlap, i.e. two selections may lead to an illumination of two different portions of the sample which are not completely spatially distinct. An A-scan corresponds to each selection by the optical element of a portion of the sample, e.g. an A-scan in an OCT image of a portion of the sample selected by the optical element. Thus, when a new selection is made in the optical element, a new A-scan is obtained.

This selection of a portion of the sample by the optical element may be done mechanically, for example considering the optical element as comprising a turning mirror that can direct the sample light signal coming from the sample arm towards a specific portion of the sample. The sample light signal can be oriented moving, e.g. rotating, the mirror itself, for example along X or Y direction, both perpendicular to the propagating direction of the sample light signal coming out of the sample arm, till the desired portion of the sample is illuminated.

Alternatively, the sample light signal coming out the sample arm may be moved on the sample to select a desired portion using an acousto-optic device, and therefore the portion of the sample to be illuminated may be selected changing a voltage or current value fed to the optical element. Any optical device apt to change the position of a sample light signal over a sample can be used as optical element as well.

The second arm of the interferometer, the reference arm, has a function as in standard SS-OCT system and outputs a reference light signal towards a reference reflector.

The sample and the reflector reflect light back into the two arms of the interferometer generating a sample returning signal and a reference returning signal, respectively.

Selected a portion of the sample to be illuminated, a sweeping of the light source is performed, that is, a tuning of the wavelength of the coherent light signal emitted by the source is performed, where the wavelength of the coherent light signal is changed within Δλ to for a sweeping time ΔT. The sweeping is performed keeping fixed—e.g. always in the same position—the beam of the sample light signal coming out of the sample arm, i.e. always impinging the same selected portion of sample for the whole sweeping duration. This sweeping corresponds to the generation of a single A-scan. During the interval ΔT, the light emitted by the source changes its wavelength from a minimum to a maximum.

In the present invention, during the sweeping, the wavelength of the coherent light signal is changed, but it is not always increasing as depicted in FIG. 1. In the present invention, the sweeping time ΔT is divided in several sub-intervals, at least two sub-intervals. In each of these sweeping sub-intervals, all belonging to the same sweeping, that is, all concurring to the realization of the same A-scan (i.e. all concurring to the formation of an OCT image of the same portion of the sample in depth), the wavelength of the coherent light signal is varied, preferably—but not necessarily—linearly.

In each sub-interval, the wavelength λ of the light source signal is varied, within the range defined by the overall minimum and maximum (but not necessarily reaching them), in such a way that the wavelength of the coherent light signal at one instant within the (i+M)ₜₕ sub-interval (where i and M are integers) has the same value which it had at a different instant during the iₜₕ interval, that is:

λ in the iₜₕ sub-interval at time t₁=λ in the (i+M)ₜₕ sub-interval at time t₂

There could be many “points” (e.g. instants of time or even time intervals) when the light source signal has the same wavelength both in the iₜₕ and in the (i+M)ₜₕ sub-interval. Additionally, if there are N>2 sub sweeping intervals, there might be an instant in the first sub-interval when the wavelength of the coherent light signal is identical to the wavelength of the coherent light signal at an instant in the second sub-interval which is also identical to the wavelength of the coherent light signal at an instant in the third sub-interval and so on, e.g.:

λ in the iₜₕ sub-interval at time t₁, t₂, t₃. . . =λ in the (i+M)-th sub-interval at time tₖ, tₖ₊₁, t_{k+2 . . .} =λ in the (i+M+L)-th sub-interval at time tₘ, tₘ₊₁, tₘ₊₂ . . .

where M, i, k, and L are integers.

The sweeping is thus divided in N sub-sweepings in which the wavelength of the coherent light signal has a given behaviour. The duration Δtᵢ of each sub-sweeping interval, for example in the number of N, where i=1 . . . N integer, is such that Σ₁^{N}Δtᵢ=ΔT.

In this way, the width of the range in which the wavelength of the light source signal has to be tuned can be smaller than in the situation of FIG. 1, but the same result is achieved in term of speed and resolution. The wavelength variation of the coherent light signal emitted by the source is divided in “sub variations” each requiring a smaller range. This does not affect the resolution of the system, as detailed below.

It is to be underlined that the light source in the SS-OCT system is a single light source performing the sweeping in the manner above outlined. In other words, the sweeping including the sub-intervals is generated by a single laser source, the wavelength of which is modulated in each sweeping sub-interval.

This coherent light signal as mentioned travels in the interferometers and generates the reference light signal and sample light signal exiting the sample reference and sample arm. These two signals, in turn, are reflected by the reference reflector and the sample, respectively, generating a reference and sample returning signals travelling back in the reference arm and the sample arm.

The two returning signals generate an interference signal, or beat signal, which is detected. The detector can be for example a photodetector. This interference signal which is detected includes the interference signal also generated by the sample light signals generated by the at least two coherent light signals coming from the laser source and impinging the sample and having the same wavelength.

The fact that the sweeping interval is divided in sub-intervals, having a temporal duration of Δt, without a constant increase of the wavelength of the coherent light signal in the whole sweeping interval having a duration of ΔT as previously defined, does not affect the resolution of the final image, because for the interference signal only the difference in path between the interfering signals is relevant, not the absolute value of the wavelengths. Without being bound by theory, it can be said that only the absolute value of the wavelength difference matters in generating the interference signal.

The A-scan for the selected portion of the sample illuminated for the duration of the sweeping is obtained using both the coherent light signals within the same sweeping and having the same wavelength, and in particular the interference signal (or beat signal) generated by both the corresponding sample returning signal of the two coherent light signal having the same wavelength is used to obtain the A-scan. It is to be understood that the same wavelength of the coherent light signals is present when the two light signals are emitted (at different times) at the source. That is, when “light signals having the same wavelength” means “light signals that have the same wavelength when they are emitted by the light source”. E.g. just outputted.

In the above mentioned first and second aspect, the invention may include the following characteristics, either in combination or as alternatives.

Preferably, sweeping the source for a time interval ΔT, includes dividing the sweeping in N, where N≥2, sub-sweeping intervals, wherein in each sub-sweeping interval, for a portion thereof, the wavelength of the coherent light signal varies with time substantially identically to the previous sub-sweeping step or varies with time opposite to the previous sub-sweeping step.

The term “opposite” is interpreted in the context of the present application as a trend indicator of the variation of the wavelength in a range of subscales. For example, if a sub-sweeping interval the wavelength of the coherent light signal increases in a subsequent sub-sweeping interval, the wavelength of the coherent light signal decreases, but not necessarily decreases at the same rate with which the wavelength increases in the previous sub-sweeping interval.

The detected interference signal generated by the sweeping, in all the N sub-sweeping intervals, is used to obtain the same A scan. Thus the same A scan may include interference signal generated by using the sample returning signals of several coherent light signals all having the same wavelength. The sweeping in the sub-interval is performed all for the same selection in the optical element.

The coherent light signal, as said, in each sub-sweeping interval, portion of the total sweeping time ΔT, may vary from a minimum to a maximum independently from the previous or subsequent sub-sweeping interval, as long as there are at least two points (e.g. time instants) during the whole sweeping time where the coherent light signal reaches the same wavelength value. Preferably, for a portion of each sub-sweeping interval, the coherent light signal wavelength has the same behaviour with respect to time, i.e. it has the same values, which are reached in the previous or subsequent sub sweeping interval. For example, if f(t) is the value of the wavelength of the coherent light signal as a function of the time, there is preferably a first time interval Δt, belonging to the i-th sub-sweeping interval and a second time interval Δtᵢ₊₁ belonging to the (i+1)th sub-sweeping interval for which

$$f ( t ) for t ∈ Δ {t}_{i} = \pm f ( t ) + C for t ∈ Δ {t}_{i + 1}$$

where C is a constant and i+1 N. The meaning of the equation is that for all instants t within time interval Δt, belonging to the i-th sub-sweeping interval, the behaviour of the wavelength over time is substantially identical, or opposite, to the behaviour of the wavelength over time for all instants t within time interval Δtᵢ₊₁ belonging to the (i+1)-th sub-sweeping interval, apart from a constant C.

In other words, the wavelength in the i-th sub sweeping interval defines a curve function of time. A portion of this curve is reproduced in the subsequent (i+1)-th sub sweeping interval, or its opposite (i.e. the opposite of the function, −f(t)). The constant C may vary in each sub sweeping interval.

It is to be understood that f(t) and constant C are such that the frequency has always a positive value.

The identity in f(t) is of course not a mathematical identity. The emission of a wavelength and the tuning of the signal are bound to tolerances of the apparatuses used and therefore the “identity” is within the above mentioned tolerances. These tolerances are preferably <20% for each point of the curve, preferably <10%, more preferably <5%, even more preferably <2%.

Applicants have realized that “positive” or “negative” frequencies' differences substantially lead to the same result when the interference signal is then processed, e.g. the beat signals stay unchanged regardless of whether the coherent light signal increases its wavelength or decreases it (in substantially the same way). In other words, the detected interference signal remains unchanged if the wavelength variation is substantially inverted. Only the absolute value of the wavelength difference may matter in generating the interference signal.

Preferably, elaborating the detected interference signal involves excluding a region of the above-mentioned signal around the time when the N−1 sub-sweeping interval ends and the N sub-sweeping interval starts.

Around the time when the wavelength behaviour as a function of time changes, for example from an increasing behaviour to a decreasing behaviour, the resulting interference signal might be not usable to obtain a proper OCT image (the same A scan). Those times, or also the neighbourhood of these times, of “behaviour changes” might be removed from the overall interference signal and not elaborated further.

Preferably, these portions which are deleted from the detected interference signal correspond to regions where the wavelength of the coherent light signal is at about its maximum or at about its minimum.

Preferably, all the sub-sweeping intervals have a substantially identical sub-sweeping duration Δt≤ΔT/2.

The total sweeping duration ΔT is preferably divided in N sub sweeping intervals all having the same duration Δt, so that Σ₁^{N}Δtᵢ=NΔt=ΔT. Due to the fact that the overall time of the sweeping phase is fixed and depends on the application, the duration of the sub sweeping intervals determines the number N of intervals. Preferably, N is not too big in order to avoid to remove many portions of the detected interference signal.

Preferably, the behaviour of the wavelength of the coherent light signal over time in each sub sweeping signal is the same, i.e. the wavelength behaviour over time is substantially periodical with period Δt.

Preferably, sweeping the swept source for a time interval ΔT includes sweeping the swept source for a time interval shorter than 10 μs, preferably shorter than 1 μs. More preferably, ΔT is shorter than 100 ns.

ΔT, the duration of an A-scan, is preferably very “quick”. However, in order to obtain an acceptable resolution in Z of the OCT image, and at the same time having a scan which is fast enough, preferably the time allotted for each sweeping is in the above claimed range.

The sub-sweeping intervals are preferably shorter than 50 ns each. More preferably, they are longer than ΔT/6. Preferably, they are shorter than ΔT/2.

Preferably, the method includes: dividing the sweeping in N, where N sub-sweeping intervals, providing the (i−1)-th sub-sweeping interval having a duration Δtᵢ₋₁ with the wavelength of the coherent light signal having the following behaviour:

λᵢ₋₁(t)=f(t) where f(t) is a monotone function between t₁ and t₂, where t₁ and t₂∈Δtᵢ₋₁; and

providing the i-th sub-sweeping interval having a duration Δtᵢ with the wavelength of the coherent light signal having the following behaviour:

λᵢ(t)=−f(t)+C where C is a constant, between t₃ and t₄ where t₃ and t₄∈Δtᵢ.

Alternatively, the method includes: dividing the sweeping in N, where N sub-sweeping intervals, providing the (i−1)-th sub-sweeping interval having a duration Δtᵢ₋₁ with the wavelength of the coherent light signal having the following behaviour:

λᵢ₋₁(t)=f(t) where f(t) is a monotone function between t₁ and t₂, where t₁ and t₂∈Δtᵢ₋₁; and providing the i-th sub-sweeping interval having a duration Δtᵢ with the wavelength of the coherent light signal having the following behaviour:

λᵢ(t =f(t)+C where C is a constant, between t₃ and t₄ where t₃ and t₄∈Δtᵢ.

Therefore, in this embodiment, the behaviour of the wavelength over time in two adjacent sub-sweeping interval is the same (f(t) is the same in both interval). C might also be equal to zero.

Preferably, for at least a portion of each sub-sweeping interval, the wavelength behaviour over time is a monotonous function of time. Thus, depicting the wavelength as a curve function of time, each sub sweeping interval includes a portion of the same curve, or its opposite, “shifted in time”, which is monotone for a time interval. Preferably, this monotone portion of curve is present in all sub sweeping intervals. λᵢ₋₁(t) indicates the value of the wavelength of the coherent light source in the interval i−1, while λᵢ(t) indicates the value of the wavelength of the coherent light source in the interval i, where i is an integer and i=1 . . . N.

More preferably, all the sub sweeping intervals have equal sub sweeping duration Δt and λᵢ₋(t)=λᵢ(t)+C where C is a constant for the whole duration of the sub sweeping interval.

Alternatively, all sub sweeping intervals have equal sub sweeping duration Δt and λᵢ₋₁(t)=−λᵢ(t)+C where C is a constant for the whole duration of the sub sweeping interval.

Preferably, the behaviour of the wavelength in all sub sweeping intervals is the same, or its opposite. Again, the definition of “the same” or “identical” refers to an identity within the above mentioned tolerances intrinsic of the apparatus. The same behaviour of the wavelength considered as a curve in a sub sweeping interval is copied and shifted in time to the next sub sweeping interval, or it is copied, the opposite is made, and then shifted.

Even more preferably, f(t) is a substantially linear function.

The wavelength is preferably a linear function of time and it is divided in linear segments, a segment for each sub sweeping interval. Preferably, the overall number of segments can be ascending or descending (e.g., they may have all positive or all negative derivative), or preferably could be alternate (i.e. some ascending and some descending).

For example, preferably, the wavelength in each sub sweeping interval has the following form:

λᵢ(t)=mt+aᵢ where i=1 . . . N and aᵢ is a constant sub-sweeping interval dependent.

In each other sub sweeping interval k, where k=1 . . . N with k the wavelength changes as:

$${λ}_{k} ( t ) = m t + {b}_{k} or {λ}_{k} ( t ) = - m t + {c}_{k}$$

Where bₖ and cₖ are constants sub-sweeping interval dependent. Thus the slope m of the linear curve stays the same or becomes its opposite. The linear curves are not strictly parallel (or opposite) in the mathematical meaning of it, that is, the value m is the same in all intervals not absolutely, but within a tolerance. Preferably, from one sub-sweeping interval to the other there can be a difference in the m value of maximum 20%, preferably lower than 10%, more preferably lower than 2%.

Preferably, all sub-sweeping intervals have identical sub-sweeping duration Δt and the wavelength of the coherent light signal is a substantially periodic function with period Δt or 2 Δt.

The wavelength vs. time behaviour could be for example that of a sawtooth wave. In this case, between a tooth of the saw and the neighbouring one, preferably the laser is switched off. The time interval in which the laser is off corresponds to a region in the interference signal that is to be discarded.

Alternatively, it could be a triangular wave. The triangle defined by the wave is preferably isosceles.

Preferably, the method includes the step of dividing the sweeping in N sub-sweeping intervals, wherein N can range from a minimum of 2 to a maximum of 15. More preferably, N can range from a minimum of 2 to a maximum of 8. Even more preferably, N can range from a minimum of 4 to a maximum of 6. The maximum number of sub-sweeping intervals depends on what is considered to be an acceptable noise level which comes from the discontinuities in the interference signal. These discontinuities, which generally are generated in correspondence to portions of a sub sweeping intervals wherein the wavelength reaches its minimum and/or its maximum values, are preferably removed before elaborating the interference signal.

Coherent light sources with a tuning speed lower than 50 nm/μs are commercially available, showing a typical tuning range around 100 nm. In order to raise the scan speed, special optical materials allow it, but they have smaller tuning ranges, typically lower than 20 nm. Therefore, the preferred number of sub-sweeping intervals is a compromise between the “small-bandwidth” generally available in tunable sources and the amount of interference signal to be discarded, and it is preferably comprised between 2 and 15, more preferably between 2 and 6.

Preferably, the method comprises providing a light source having a spectral bandwidth narrower than 40 nm. More preferably the spectral bandwidth is narrower than 30 nm, even more preferably, the spectral bandwidth is narrower than 25 nm.

Preferably, the light source is a tunable laser source including a liquid crystal tunable element.

The liquid crystal is preferably the tunable element that allows the wavelength change of the coherent light source.

Preferably, the light source is a laser source having a cavity. The cavity is limited by mirrors. Preferably, one of the mirrors is a partially reflective mirror and the other is a high reflectivity mirror. The cavity includes a gain medium and an optical tunable filter. The optical tunable filter includes a liquid crystal.

As known, for the gain medium to amplify light, it needs to be supplied with via pumping. The energy is typically supplied as an electric current or as light at a different wavelength. Light from the gain medium bounces back and forth between the mirrors, passing through the gain medium and being amplified each time. The light also passes through the tunable optical filter. The partially transparent mirror allows some of the light to escape through it. Therefore, depending on the characteristics of the optical filter, for example its refractive index, the wavelength of the light which escapes the cavity through the partially transparent mirror may vary. Changing the characteristics of the tunable optical filter changes the wavelength of the light outputted by the laser source.

The optical filter of the invention has a given bandwidth or free spectral range, i.e. it can be tuned from a minimum to a maximum value of refractive index by applying an electromagnetic field to it.

Due to the fact that the wavelength of the light in the cavity varies because the optical filter can be tuned, also the partially transparent mirror has preferably a given free spectral range. Preferably, the free spectral range of the partially transparent mirror is the same or substantially the same of the free spectral range of the optical filter. In this way, the linearity of the output of the laser source can be obtained and the simultaneous lasing at two or more wavelengths is substantially prevented. Preferably the free spectral range of the mirror and of the optical tunable filter is narrower than 40 nm, more preferably narrower than 30 nm, more preferably wider than 20 nm.

The tuning of the wavelength of the output of the laser source, i.e. the wavelength of the coherent light signal, thus depends on the refractive index of the tunable optical filter. However, a change in the wavelength of the coherent light source in the present invention is preferably not caused by the standard electro-optic phenomenon which is related to Frederiks effect, i.e. reorientation of molecule director n in low frequency electric field caused by anisotropy of dielectric susceptibility. This effect, the well-known common effect in Liquid Crystals, causes too slow a variation, (e.g. having a response time of the order of a millisecond), of the material refractive index for the needs of an OCT system. The effect used in the present invention to obtain a variation of the wavelength of the Liquid Crystal in the tunable optical filter in the cavity of the laser source is the NEMOP effect (Nanosecond Electrically Induced Modification of Order Parameters of the liquid crystal). The liquid crystal can be of any type carrying a positive or negative dielectric and magnetic anisotropy and may include several kind of additives like, but not limited to: polymeric compounds, nanoparticles, strongly polar molecules.

Preferably, the tunable optical filter is an etalon (also named Fabry-Pérot filter).

The tuning of this material is preferably performed by applying an external electromagnetic field across the liquid crystal, for example via electrodes.

For example, the liquid crystal in the laser of the invention fills a gap between two optically transparent slabs (preferably glass), wherein said gap has a width which is narrower than 100 μm, preferably narrower than 50 μm, even more preferably narrower than 30 μm. On the other hand, the width of the gap is preferably wider than 10 μm. In general, the narrower the width of the gap between two optically transparent slabs, the broader the Free Spectra Range of the resulting tuneable filter. At the same time, the gap has preferably a minimum width, so that the liquid crystal is able to penetrate between said two optically transparent slabs, filling the gap.

The liquid crystal is preferably positioned between two electrodes, for example thin films of low resistivity, high transparency TCO (transparent conductive oxide) material. These conductive layers preferably face one another inside the cell and are separated by a suitable gap filled up by the chosen material. The cell may be sealed by means of a gasket containing size-controlled microparticles to ensure uniform distance. Further, a highly reflective dielectric multilayer is preferably deposited on top of at least one, preferably on top of each, of the TCO to ensure a Fabry Perot behavior. It is to be understood that the meaning of “on top” is equal to “in contact with a surface of”, being the orientation of the liquid crystal cell arbitrary. The reflectivity of the high reflectivity dielectric multilayer is preferably greater than 95% in order to ensure a narrow line width output of the signal from the cavity.

The electrodes are connected to a signal generator so that a signal can be applied to the electrodes to generate an electromagnetic field.

According to an embodiment, the cell comprises, from top to bottom (top and bottom are used to describe a succession of layers, the physical orientation of the cell can be arbitrary): quartz or glass substrate, a layer of Indium Tin Oxide (ITO) conductive and transparent to the wavelengths travelling in the cavity (this define the electrode), a dielectric multilayer having a high reflectivity and including two layers, a low refractive index one (e.g. SiO₂) and a high refractive index one (e.g. TiO₂), the liquid crystal and then again dielectric multilayer, ITO and glass or quartz substrate. The position of the electrode and the multilayer can be exchanged to modify the reflectivity in the wavelength range of interest.

The external electro-magnetic field is preferably applied in switch-on and switch-off configurations. For example, in a sub-sweeping interval, the electromagnetic field is applied to the LC and, changing from one sub-sweeping interval to the next, it is switched off. Alternatively, it can be varied quickly. Typical raise and fall times of the electromagnetic field in this on/off behavior are of about 5-10 ns. It is to be noted that the liquid crystal response due to NEMOP effect shows very fast response time, typically much lower than 100 ns.

The typical cell thickness range in order to obtain laser source tunability in the desired range, for example in a range wider than 20 nm, is preferably between 10 and 50 microns, more preferably between 15 and 40 microns, even more preferably between 20 and 30 microns. The thickness of the cell is substantially the thickness of the liquid crystal because the thickness of the dielectric multilayer is relatively small, e.g. it can be comprised between 1 micron and 5 micron, for a thicker cell, e.g. having a thickness smaller than 100 micron, it can be comprised between 1 and 10 micron.

It is to be considered that in this configuration, the liquid crystal could be replaced by a thin slab of electro-optic material with high electro-optical coefficient (>30 pm/V), like lithium niobate (LiNbO₃) or rubidium tytanil phosphate (RTP): the slab thickness will be lower with respect to the needed liquid crystal thickness because of the higher refractive index of the electro-optic crystal, in a way that the optical path travelled by the light within the Fabry Perot is the same in both cases.

In order to obtain a linear tunability, the signal generator energizes the electrodes which apply a driving voltage to the liquid crystal (LC) in the optical filter. The driving voltage is preferably higher than 0.1 kV, preferably comprised between 0.2 kV and 2 kV, more preferably between 0.5 kV and 1 kV. Varying the voltage linearly, the refractive index of the LC is varied linearly as well changing the transmission characteristic of the Fabry-Perot filter.

The Applicants have understood that by applying a voltage difference to the liquid crystal for a driving time shorter than 1 microsecond, relatively “slow” effects causing the liquid crystal refractive index changes are prevented or reduced. This holds also in the case of multiple repeated applications of a voltage difference for a plurality of driving times (shorter than 1 microsecond), provided said plurality of driving times are generated with a repetition rate comprised between 100 KHz and 100 MHz, which corresponds to a repetition time comprised between 0.01 milliseconds and 0.01 microseconds.

The term “slow” is herein construed as an effect having a typical response time of the order of a millisecond, such as for example the thermal and/or electrical driven reorientation of the molecular axis of the liquid crystal molecules.

Those slow effects can cause a strong liquid crystal refractive index changes (Δn>0.1) when driven to a maximum frequency of 10 KHz. On the other hand, the refractive index change caused by those “slow” effects decreases when the driving frequency exceeds 10 kHz. In particular, when the voltage difference is applied to the liquid crystal for a driving time shorter than 1 microsecond, the contribution to the liquid crystal refractive index change of any “slow” effect is lower or even much lower than the liquid crystal refractive index change due to the NEMOP effect, which can be as large as to produce a liquid crystal refractive index reversible change Δn greater than 0.01 (at about 0.5 KV of driving voltage difference). Again, this holds also in the case of a multiple repeated applications of a voltage difference for a plurality of driving times (shorter than 1 microsecond), provided said plurality of driving times are generated with a repetition rate comprised between 100 KHz and 100 MHz.

Repetition rates even higher than 100 MHz, i.e. in the GHz range or higher, can be also envisaged with a suitable doping of the liquid crystal.

Preferably, the interference signal is further elaborated, for example using a Fast Fourier Transform (FFT). The peaks in frequency that can be found in the FFT gives the desired z information of that portion of the sample that is illuminated during the A-scan by the coherent optical signal. Due to the fact that, in a sweeping, more than a reflection can take place, more than a peak can be detected, giving information on the position in z of more than a structure.

### BRIEF DESCRIPTION OF THE DRAWINGS

The present invention will be better understood with non-limiting reference to the appended drawings, where:

FIG. 1 represents a behavior of the variation of the wavelength (λ) over time (t) in a light source according to the prior art;

FIG. 2 is a schematic representation of a SS-OCT system according to the invention;

FIG. 3A is a detail of the system of FIG. 2;

FIG. 3B is a detail in enlarged view of FIG. 3A;

FIG. 4 represents as a solid line a first embodiment of a behavior of the variation of the wavelength (Δλ), expressed in nanometers, over time (t) in a light source of system of FIGS. 2 and 3A-B according to the present invention, the shown dotted line represents the signal of FIG. 1;

FIG. 5A represents the amplitude (A) in arbitrary units of the resulting interference signal over time (t), expressed in microseconds, when the signal of FIG. 1 is used to illuminate a portion of a sample according to the prior art;

FIG. 5B represents the amplitude (A) in arbitrary units of the resulting interference signal over time (t), expressed in microseconds, when the signal of FIG. 4 is used to illuminate the same portion of the same sample of FIG. 5A according to the invention;

FIG. 5C represents the superposition of FIGS. 5A and 5B;

FIG. 6 represents a second embodiment of a behavior of the variation of the wavelength (Δλ), expressed in nanometers, over time (t) in a light source of system of FIGS. 2 and 3A-B according to the present invention, the shown dotted line represents the signal of FIG. 1 according to prior art;

FIG. 7A represents the amplitude (A) in arbitrary units of the resulting interference signal over time (t), expressed in microseconds, when the signal of FIG. 1 is used to illuminate a portion of a sample according to the prior art;

FIG. 7B represents the amplitude (A) in arbitrary units of the resulting interference signal over time (t), expressed in microseconds, when the signal of FIG. 6 is used to illuminate the same portion of the same sample of FIG. 7A according to the invention;

FIG. 7C represents the superposition of FIGS. 7A and 7B;

FIG. 8A represents the amplitude (A) in arbitrary units of the resulting interference signal over time (t), expressed in microseconds, when the signal of FIG. 1 is used to illuminate a portion of a sample according to the invention, where two reflections are present;

FIG. 8B represents the amplitude (A) in arbitrary units of the resulting interference signal over time (t), expressed in microseconds, when the signal of FIG. 6 is used to illuminate the same portion of the same sample of FIG. 8A according to the invention;

FIG. 8C represents the superposition of FIGS. 8A and 8B;

FIG. 9A shows the amplitude (A) in arbitrary units of the fast Fourier transform (FFT) over frequency (f) in arbitrary units for the interference signal of FIG. 8A;

FIG. 9B shows the amplitude (A) in arbitrary units of the fast Fourier transform (FFT) over frequency (f) in arbitrary units for the interference signal of FIG. 8B; and

FIG. 9C shows the superposition of FIGS. 9A and 9B.

### DESCRIPTION OF PREFERRED DETAILED EMBODIMENTS OF THE INVENTION

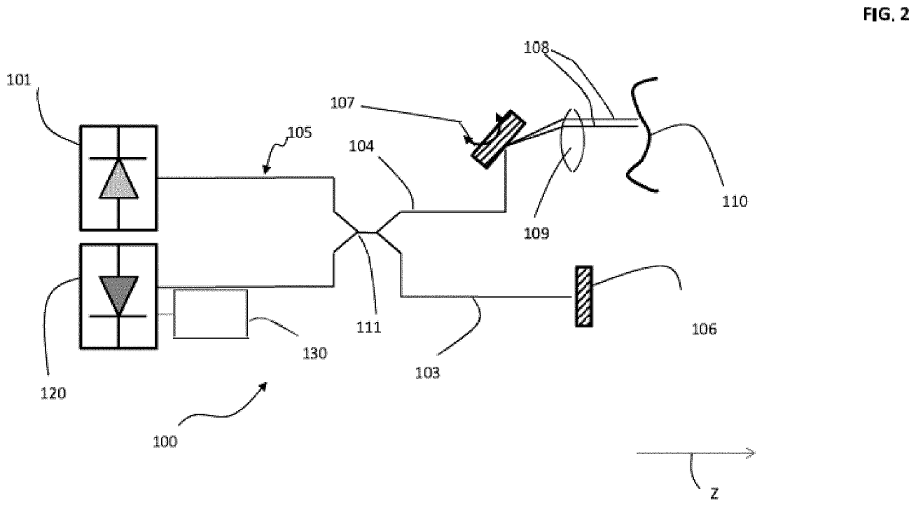

In FIG. 2, an optical coherence tomography scanner 100 for SS-OCT is illustrated. The scanner is used to illuminate a sample 110, a typical sample being tissues at the back of the human eye.

The scanner 100 includes a spatially coherent source of light, 101. This source is preferably a Swept laser Source.

Further, the scanner includes an interferometer 105, for example including two arms called reference and sample arms, 103, 104 realized with optical fibers.

Light from source 101, i.e. a coherent light signal, is routed to illuminate the sample 10 via the sample arm 104 of the interferometer 105. Further, the light from source 101 illuminates a reference reflector 106 via the reference arm 103.

The scanner 100 further includes an optical element 107 positioned between the end of the sample arm 104 and the sample 110. The optical element 107 is able to scan light exiting the arm 104 on the sample 110, so that the beam of light (dashed line 108) sweeps over the area or volume to be imaged. This area or volume of the sample which is imaged at a given time by the optical element is called selected portion of the sample 110.

The direction of light propagation of the light towards the sample outputted from the sample arm defines a Z direction or depth. A plane perpendicular to it, where the sample 110 lies at least partially, defines a (X, Y) plane.

Light scattered from the sample 110 is collected, typically into the same sample arm 104 used to route the light for illumination of the selected portion of the sample 110.

Reference light derived from the same source 101 travels a separate path, involving reference arm 103. The light outputted by the reference arm 103 is reflected by a reflector 108. A reflected light from the reflector is thus travelling backwards in the reference arm 103.

These two “returning” sample and reference lights back-propagating in the sample and reference arms 103, 104 are collected. Collected sample returning light is combined with collected reference returning light, typically in a fiber coupler 111, to form interference light which is routed to a detector 120, such as a photodiode. The output from the detector 120 is supplied to a processor 130. The results can be stored in the processor.

The interference causes the intensity of the interfered light to vary across the spectrum. For any scattering point in the sample, there will be a certain difference in the path length between light from the source and reflected from that point, and light from the source traveling the reference path. The interfered light has an intensity that is relatively high or low depending on whether the path length difference is an even or odd number of half-wavelengths, as these path length differences result in constructive or destructive interference, respectively. Thus the intensity of the interfered light varies with wavelength in a way that reveals the path length difference; greater path length difference results in faster variation between constructive and destructive interference across the spectrum.

The Fourier transform of the interference spectrum reveals the profile of scattering intensities at different path lengths, and therefore scattering as a function of depth in the sample.

The profile of scattering as a function of depth is called an axial scan (A-scan). A set of A-Scans measured at neighboring locations (various selected portions) in the sample produces a cross-sectional image (tomogram) of the sample 110.

The range of wavelengths at which the interference is recorded determines the resolution with which one can determine the depth of the scattering centers, and thus the axial resolution of the tomogram.

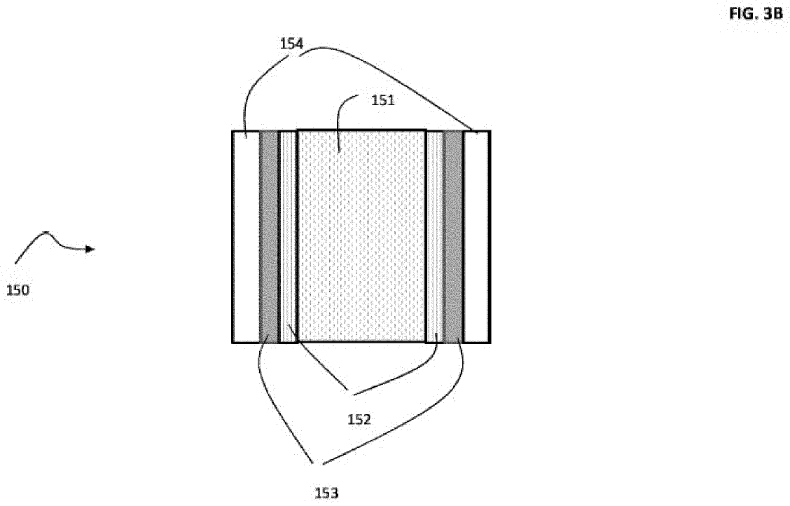

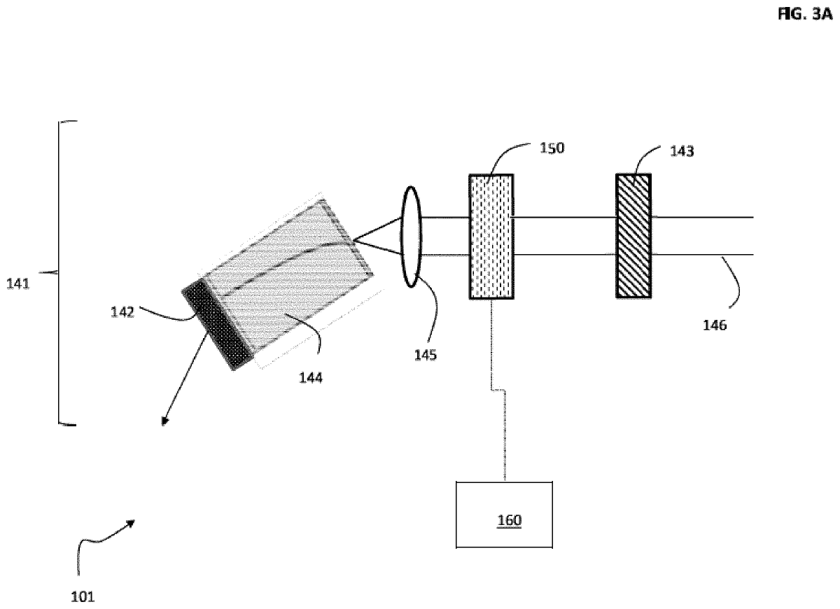

A more detailed view of the laser source 101 used in the scanner 100 according to the invention is depicted in FIG. 3A. The laser source, in order to tune the wavelength of the emitted signal, uses a liquid crystal 150 based etalon with a Free Spectral Range of 25 nm and a frequency response of around 10 MHz.

The laser source 101 includes a cavity 141 delimited by a first and a second mirror 142, 143. The first mirror 142 is a highly reflective mirror, while the second mirror 143 is a partially transparent mirror having a mirror FSR and has the function of output coupler. The output of the etalon 150 is indicated with 146 in the figure.

The cavity 141 further includes a gain medium or gain chip 144, pumped in a known way, and a collimating lens 145 to focus the light on the etalon 150. Etalon 150 is connected to a voltage generator 160.

The processor 130 connected to the laser source 101 changes the etalon driving voltage via the voltage generator 160 so that, during an A-scan, the wavelength of the coherent light signal emitted from the laser source 101 changes according to the invention.

In FIG. 3B, a more detailed view of the etalon 150 is shown in an enlarged view.

The etalon 150 includes a liquid crystal element 151. The liquid crystal element may include any of: CCN-47, MLC-20180, HNG715600-100 produced by Nematel GmbH (Germany), Merck (USA), Jiangsu Hecheng Display technology (china), respectively.

The liquid crystal element 151 is doped with a polar addictive, preferably 2, 3-dicyano-4-pentyloxyphenyl 4′-pentyloxybenzoate (DPP), CAS 67042-21-1 produced by UAB Tikslioji Sinteze, Lithuania.

More information about the used liquid crystal material can be found in “Enhanced nanosecond electro-optic effect in isotropic and nematic phases of dielectrically negative nematics doped by strongly polar additive”, published in Journal of Molecular Physics, December 2017, written by Bingxian Li et al.

Two opposite sides of the LC element 151 are coated with a high reflectivity dielectric multilayer (reflectivity higher than 95%) 152 and the resulting structure is sandwiched between two electrodes 153 attached to the voltage generator 160.

Two glass slabs then closes the etalon 150.

The voltage generator applies a suitable voltage to the electrodes 153 so that the refractive index of the LC element 151 changes. A linear voltage variation implies a linear change in the wavelength of the output 146.

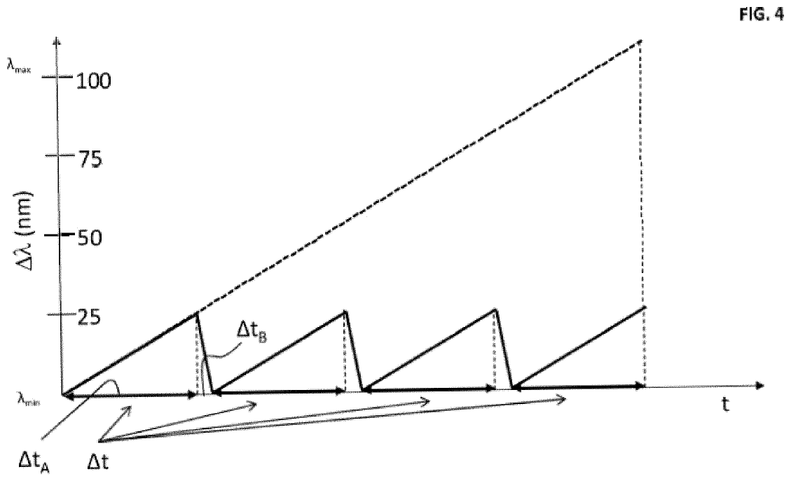

In FIG. 4, a first preferred embodiment of the sweeping for an A scan which last ΔT is shown, the sweeping duration ΔT is divided is sub intervals of equal duration Δt.

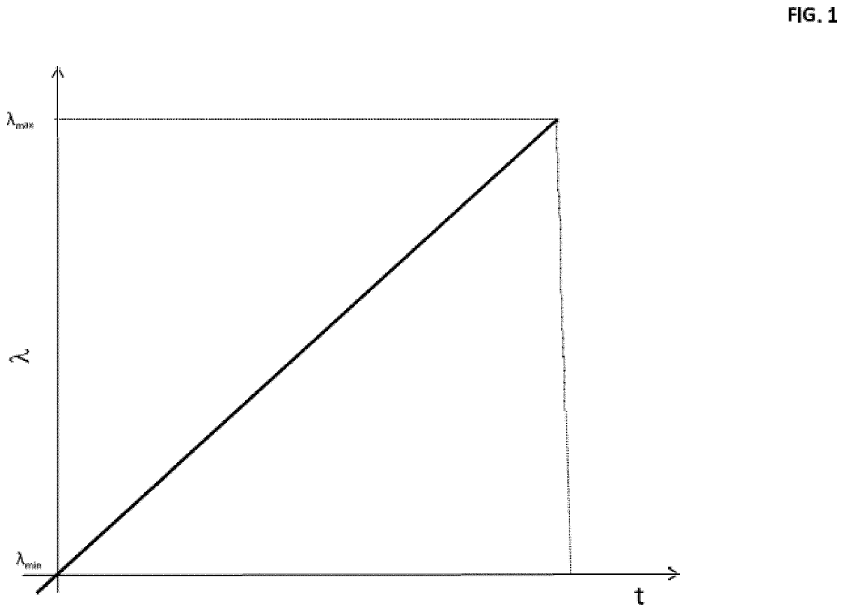

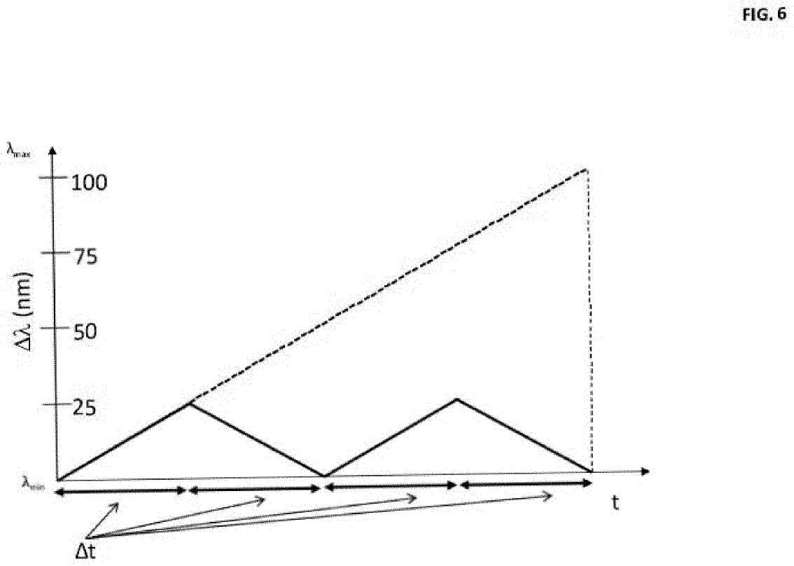

It is to be understood that the “wavelength” ordinate represents a variation from a minimum wavelength to a maximum wavelength. For practical reasons of representation, the minimum wavelength is represented as if it were the “zero” ordinate, however in reality the minimum wavelength of the coherent light signal emitted by the light source is different from zero. Thus the value shown is always (minimum wavelength)−(maximum wavelength). The same considerations applies to FIG. 1 and FIG. 6.

In this embodiment, as visible in the figure, in each of these sub intervals of duration Δt, the wavelength of the coherent light output 146 is increased linearly and monotonously for a duration Δt_{A}. Further, in the same sub sweeping interval, the wavelength is decreased linearly and monotonously for a duration Δt_{B} where preferably Δt_{B}<<Δt_{A}. The resulting wavelength behaviour of the wavelength of the coherent light signal 146 over t is a periodic function in time with period Δt=Δt_{A}+Δt_{B}. The wavelength defines substantially, if Δt_{B} <<Δt_{A}, a slightly “deformed” sawtooth function of time as represented in FIG. 4. The sawtooth scan can be made or with a very fast reset of the tuneable filter 150 if the electro-optical material is enough fast or using a beam splitter for dividing the light source in two or more portions and an optical delay line(s) to combine said portions in a sawtooth profile.

In FIG. 4, the prior art tuning of the wavelength is also shown (linear dashed curve equivalent to FIG. 1), where the wavelength linearly increase for the whole duration of the sweeping ΔT.

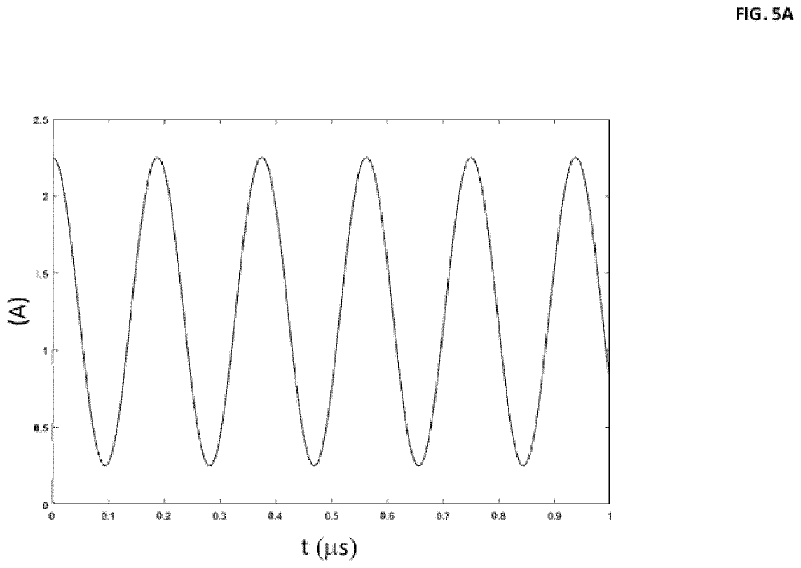

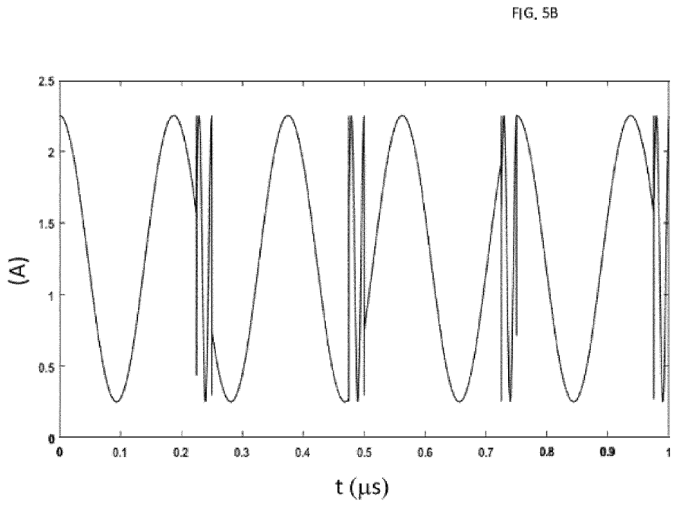

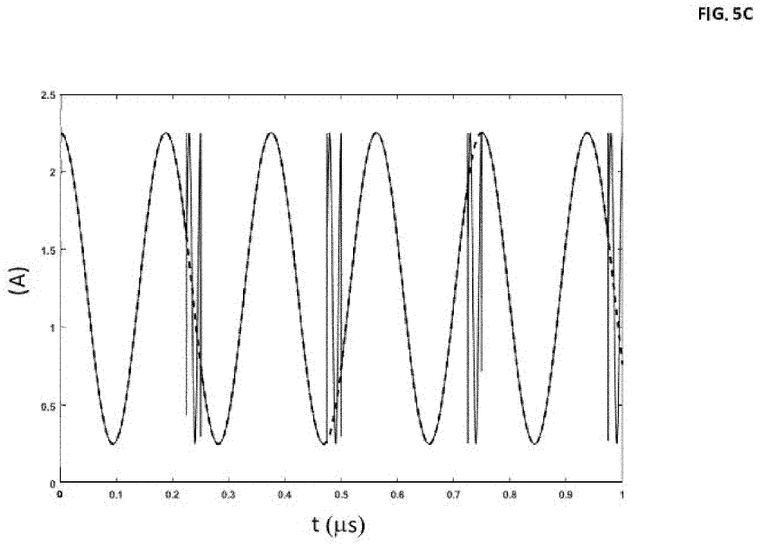

A numerical simulation of the signal from the OCT detector 120 of the interference signal obtained in case the signals (prior art and invention) of FIG. 4 is swept over the selected portion of the sample is depicted in FIG. 5A and 5B, in the prior art result in FIG. 5A and the present invention case in FIG. 5B. Further, in FIG. 5C a superposition of the two signals is made (dashed line=prior art, solid curve=present invention).

In FIG. 5A, prior art case, the interference signal is a sinusoid.

In FIG. 5B, the interference signal shows a sinusoid and some “noise portions”. It is possible to see from FIG. 5B that the interference signal in the invention presents a plurality of regions where the signal cannot be used. This portions are thus preferably discarded. These regions correspond to the portions Δt_{B} of the sub sweeping intervals. However, it can also be seen that in the remaining part of the curve (i.e. outside the discarded “noise” portions) the signal is in perfect agreement with the prior art signal, i.e. there is substantially no difference in varying the wavelength continuously from a minimum to a “high” maximum and varying the wavelength from a minimum to a much smaller maximum and repeating this change several times. This can be clearly seen in FIG. 5C where the two signals correspond perfectly outside the “noise” portions.

It can be shown that, if Δt_{B} is reduced to a minimum, the resulting portions to be discarded can be reduced as well. The smaller Δt_{B} is, the smaller the part of the resulting interference signal that needs to be not considered becomes (e.g. the discarded portions become smaller).

In FIG. 6, a second preferred embodiment of the sweeping for an A scan which last ΔT is shown, the sweeping duration ΔT is divided in sub-intervals of equal duration.

In each of these sub-intervals of duration Δt, the wavelength is varied linearly and monotonously for the whole duration Δt. However, the variation is alternatively either increasing or decreasing. In a first sub sweeping interval, the wavelength is for example increased linearly and monotonously and in the following sub sweeping interval the wavelength is decreased linearly and monotonously. The slope of the linear curve is the same albeit opposite. In other words, if in the i-th interval the slope of the segment defined by the function wavelength (t) is m, the slope of the curve in the (i+1)-th interval is −m.

This behaviour of the signal is obtained increasing with a certain speed the voltage applied to the electrodes 153, reaching a maximum, and then decreasing the voltage till the minimum at the same speed of the increase.

In FIG. 6, the prior art tuning of the wavelength is also shown (linear dashed curve equivalent to FIG. 1), where the wavelength linearly increase for the whole duration of the sweeping ΔT.

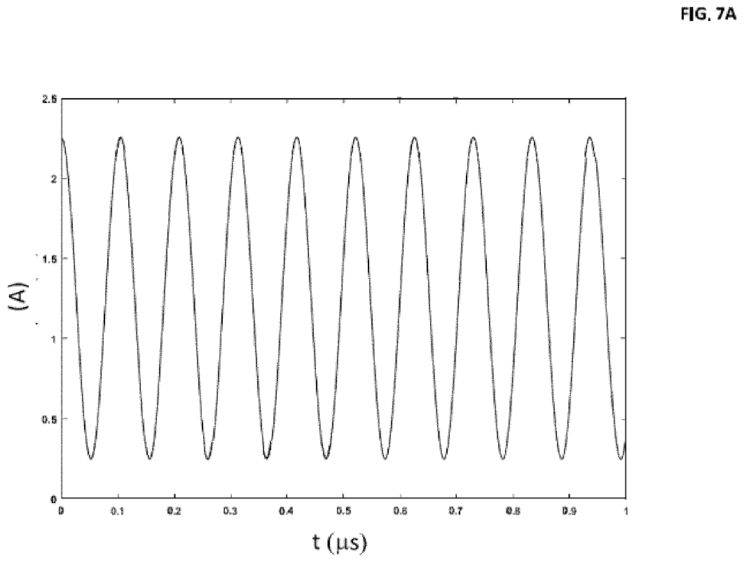

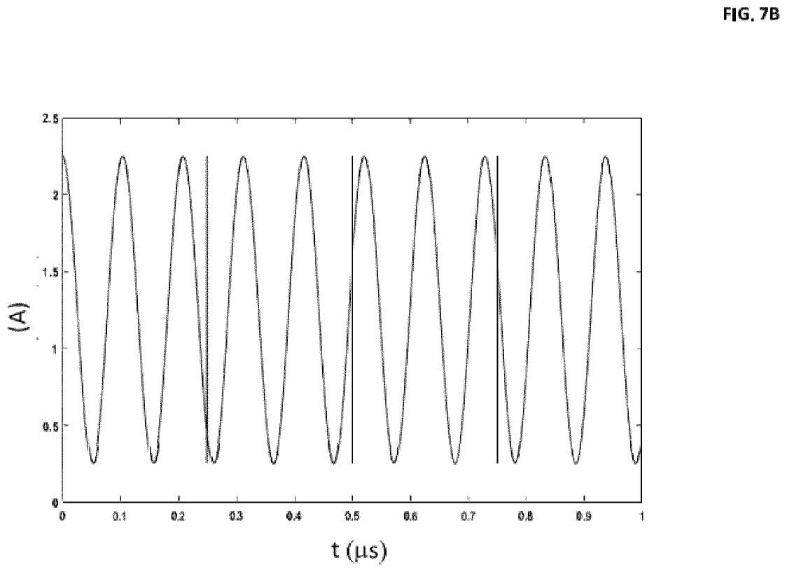

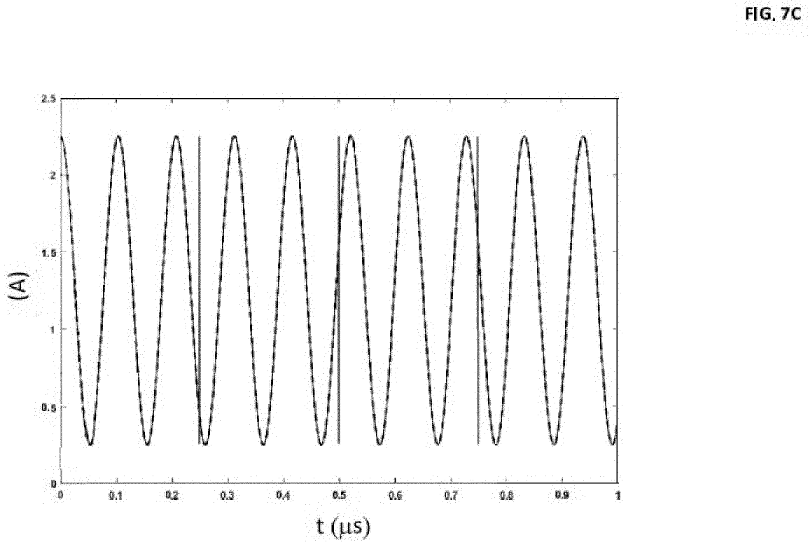

A numerical simulation of the signal from the OCT detector 120 of the interference signal obtained in case the signals (prior art and invention) of FIG. 6 are swept over the selected portion of the sample is depicted in FIG. 7A and 7B. <The prior art results are in FIG. 7A and the present invention case is shown in FIG. 7B. Further, in FIG. 7C, a superposition of the two signals is made (dashed line=prior art, solid curve=present invention).

In FIG. 7A, prior art case, the interference signal is a sinusoid.

In FIG. 7B, the interference signal shows a sinusoid and some “noise portions”. It is possible to see from FIG. 7B that the interference signal in the invention presents a plurality of regions where the signal cannot be used. These regions correspond to the boundary between one sub-sweeping interval and the next sub-sweeping interval. They also correspond to the point in which the wavelength changes behavior, from increasing to decreasing. However, it can also be seen that in the remaining part of the curve (i.e. outside the noise portions which should be discarded) the signal is in perfect agreement with the prior art signal, i.e. there is substantially no difference in varying the wavelength continuously from a minimum to a “high” maximum and varying the wavelength from a minimum to maximum and from the maximum to the same minimum, repeating this change several times. This can be clearly seen in FIG. 7C where the two signals correspond perfectly outside the “noise” portions.

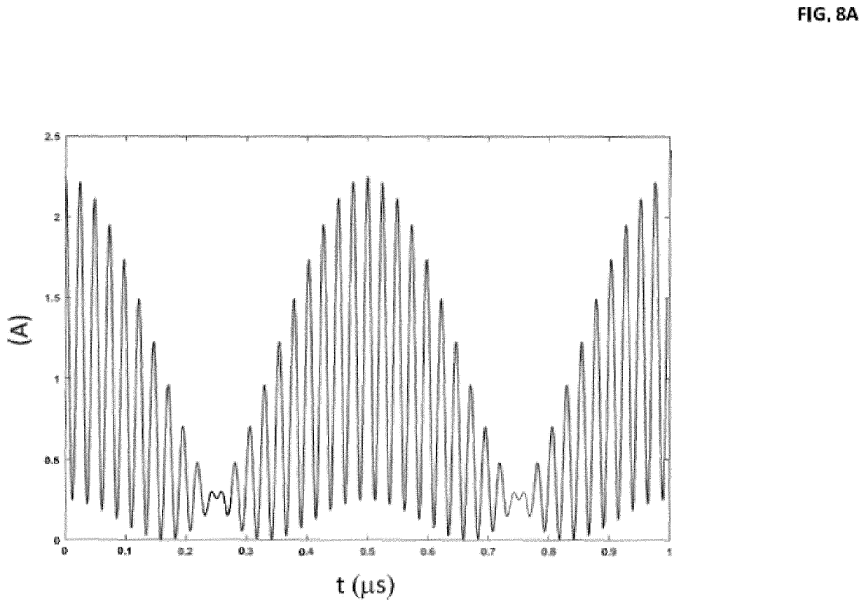

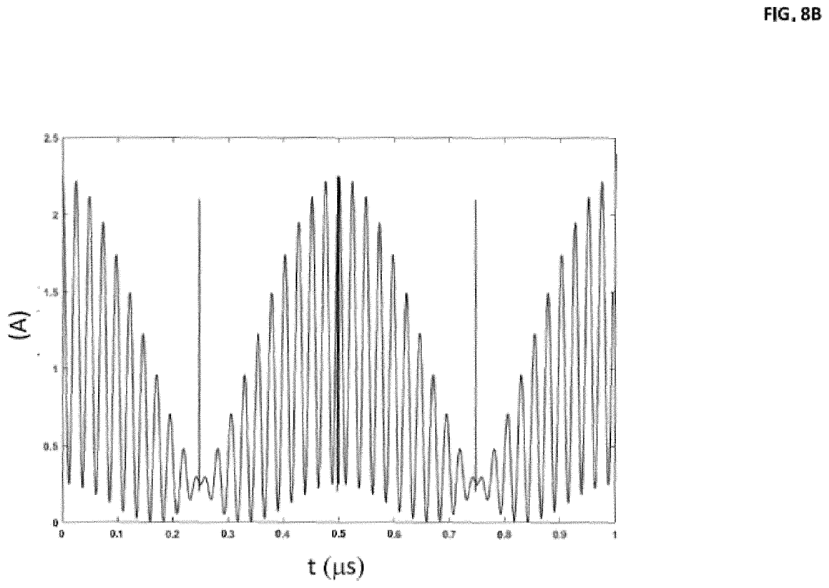

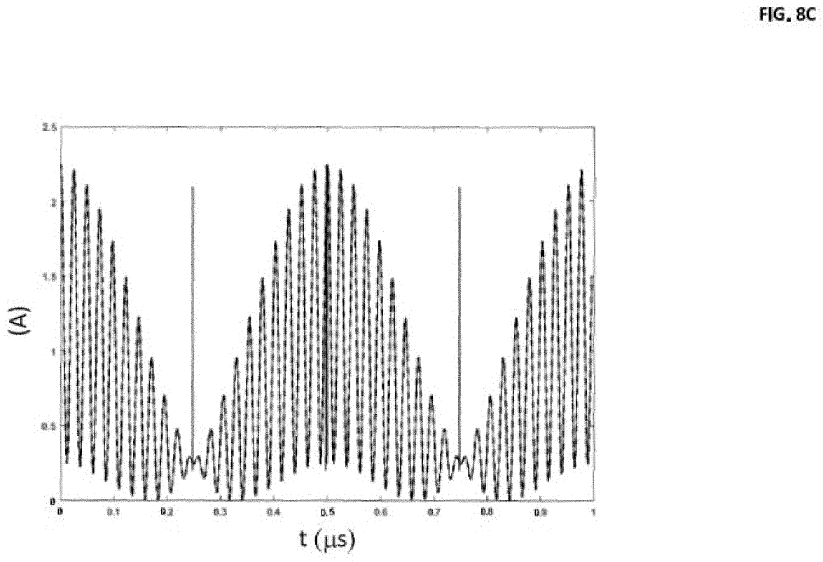

FIG. 8A-8C show the simulations results using the second embodiment sweeping signal of FIG. 6, however in this case two reflections separated by 10 μm are present in the sample.

A numerical simulation of the signal from the OCT detector 120 of the interference signal obtained in case the signals (prior art and invention) of FIG. 6 are swept over the selected portion of the sample is depicted in FIG. 8A and 8B. The prior art results are shown in FIG. 8A, and the results of the present invention case in FIG. 8B. Further, in FIG. 8C a superposition of the two signals is made (dashed line=prior art, solid curve=present invention).

In FIG. 8A, prior art case, the interference signal is a superposition of two sinusoids having different frequency. Each frequency represents a different reflection on the sample.

In FIG. 8B, the interference signal shows also two sinusoids superimposed, and some “noise portions”. It is possible to see from FIG. 8B that the interference signal in the invention presents a plurality of regions where the signal cannot be used. These regions correspond to the boundary between one sub sweeping interval and the next sub sweeping interval. They also correspond to the point in which the wavelength changes behavior, from increasing to decreasing. However, it can also be seen that in the remaining part of the curve (i.e. outside the noise portions which can be considered as discarded portions) the signal is in perfect agreement with the prior art signal, i.e. there is substantially no difference in varying the wavelength continuously from a minimum to a “high” maximum and varying the wavelength from a minimum to maximum and from the maximum to the same minimum, repeating this change several times. This can be clearly seen in FIG. 8C where the two signals correspond perfectly outside the “noise” portions.

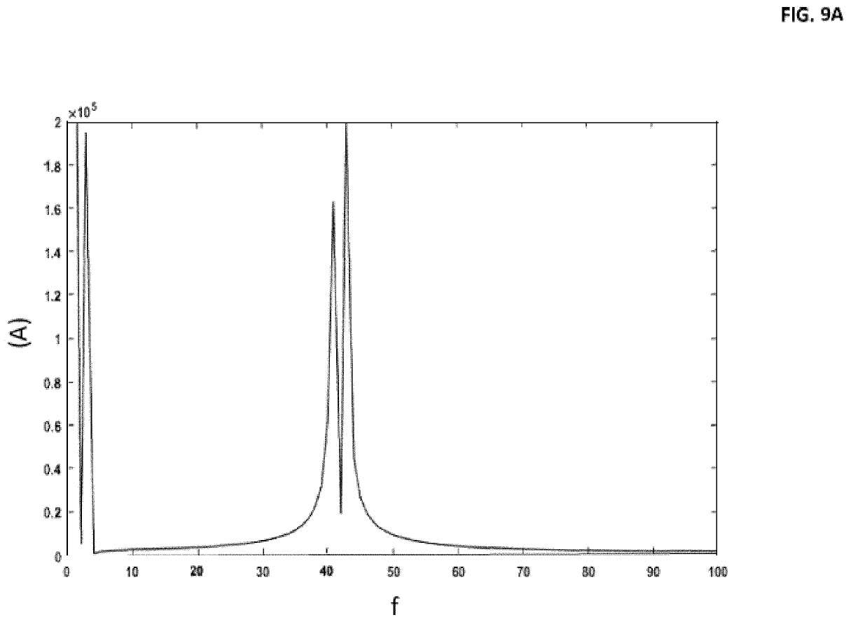

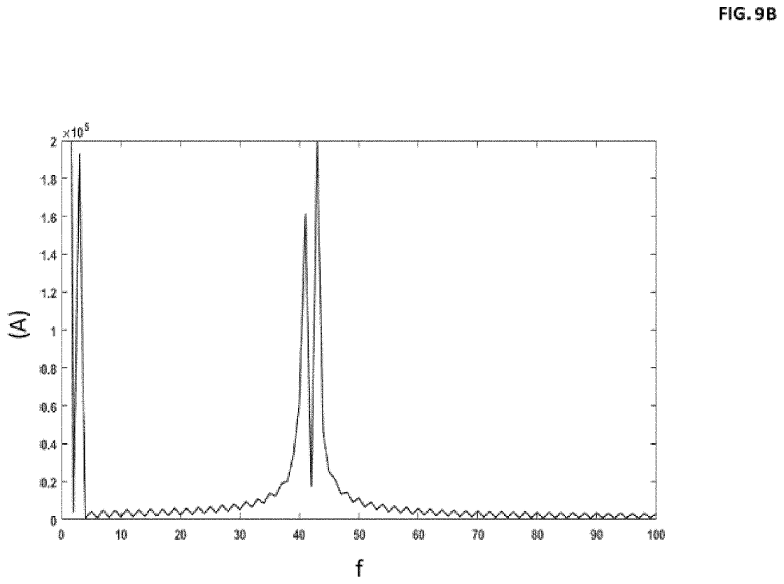

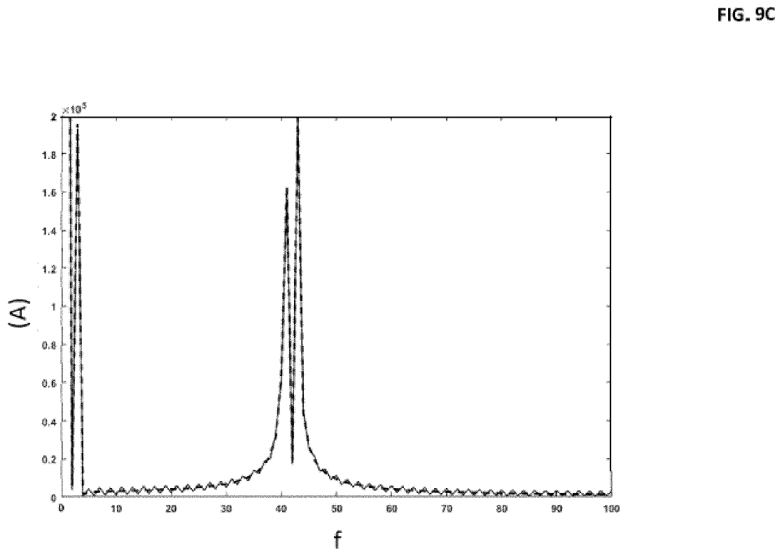

FIG. 9A-9C show the fast Fourier transform (FFT) for the interference signals of FIGS. 8A-8C (respectively) where the two reflections can be clearly distinguished, in the two cases of prior art and present invention. It is possible to see that the two spectral behaviors are very similar with only a small added noise for the present invention case.

### EXAMPLES

The laser can emit light at 1550 nm using InP based gain chip. The emission wavelength change by tuning the intra cavity tunable filter at different transmission wavelength by varying the voltage applied to the electro-optical material (in this case the electro-optical material is a thin liquid Chrystal film inside a Fabry-Perot cavity). The output of the laser is coupled at the input of an interferometer (a 2×2 in fiber coupler). At the other input arm, a fast photodiode (bandwidth around 1 GHz) is coupled and connected with a signal processor. At the end of one of the output arms the reference mirror is fixed and at the other output arm the scanning element based on a collimating lens and a scanning mirror are positioned. The length of the two output arms is preferably balanced for optimum interferometer work.

The sweeping time is set to be equal to 1 μs and it is divided in N=4 sub sweeping interval, each of 250 ns.

What is called “prior art” signal is substantially the sweeping of FIG. 1, obtained maintaining the laser source sweeping for 1 μs covering 100 nm.

The signal as depicted in FIG. 6 is obtained sweeping the laser for 250 ns increasing the output wavelength of 25 nm, then inverting the sweep for other 250 nm returning at the initial wavelength and then the previous two sweeps as described are repeated for a second time. During this 1 μs (4×250 ns), the optical element of the OCT remains fixed on the same measurement point of the sample. Voltage difference values applied to the electrodes vary between 0 and 2 kV which are enough to ensure a laser tunability of at least 20 nm, preferably at least 25 nm.

The signal of FIG. 4 is obtained sweeping the output wavelength linearly for 225 ns at a slightly higher speed covering 25 nm, then reset in 25 ns and the cycle is repeated four times (see FIG. 4). As in the previous example, during this 1 μs (4×250 ns), the optical element of the OCT remains fixed on the same measurement point of the sample.

The electrical signal from the photodiode is then amplified and sampled (in the example 10 sample per ns). The resulting 10000 samples are then Fourier transformed using a Cooley-Tukey Fast Fourier Transform (FFT) algorithm.

1-15. (canceled).

16. An optical coherence tomography analysis method, comprising:
Providing a Swept Source Optical Coherence Tomography system (SS-OCT), the SS-OCT system including:
a light source, tunable over a spectral band, that generates a coherent light signal;
an optical interferometer for dividing the coherent light signal into a reference arm leading to a reference reflector and a sample arm leading to a sample;
an optical element to selectively direct a sample light signal exiting the sample arm to a specific portion of the sample, so that for each selection in the optical element a different specific portion of the sample is illuminated;
an optical detector for detecting an interference signal generated by a combination of reference and sample returning signals from the reference arm and from the sample arm, reflected by the reference reflector and the sample, respectively;
Wherein, for the same selection in the optical element illuminating a specific portion of the sample, the method further comprises:
sweeping the light source for a time interval ΔT, so that a wavelength of the coherent light signal leading to the sample light signal illuminating the specific portion of the sample changes from a minimum wavelength to a maximum wavelength and wherein the wavelength of the coherent light signal reaches the same value between the minimum wavelength to the maximum wavelength at least twice during the sweeping;
detecting the interference signal generated by the sweeping, including portions of interference signal generated by using the sample returning signals of the at least two coherent light signals having the same wavelength;
elaborating the detected interference signal generated by the sweeping, including portions of the detected interference signal generated by using the sample returning signals of the at least two coherent light signals having the same wavelength, for obtaining an OCT image of the specific portion of the sample.

17. The method according to claim 16, wherein sweeping the light source for a time interval ΔT, includes dividing the sweeping in N, where N≥2, sub-sweeping intervals, wherein in each sub-sweeping interval, for a portion thereof, the wavelength of the coherent light signal varies with time substantially identically to the previous sub-sweeping step or varies with time opposite to the previous sub-sweeping step.

18. The method according to claim 16, wherein elaborating the detected interference signal includes excluding a region of the detected interference signal around to the time when the N−1 sub-sweeping interval ends and the N sub-sweeping interval starts.

19. The method according to claim 16, wherein all the sub-sweeping intervals have a substantially identical sub-sweeping duration Δt≤ΔT/2.

20. The method according to claim 16, wherein sweeping the light source for a time interval ΔT includes sweeping the light source for a time interval shorter than 10 μs, preferably shorter than 1 μs.

21. The method according to claim 16, further comprising:
dividing the sweeping in N, where N≥2, sub-sweeping intervals;
providing the (i−1)-th sub-sweeping interval having a duration Δtᵢ₋₁ with the wavelength of the coherent light signal having the following behaviour:
λₜ₋₁(t)=f(t) where f(t) is a monotone function between t₁ and t₂, where t₁ and t₂ ∈Δtᵢ₋₁; and
providing the i-th sub-sweeping interval having a duration Δtᵢ with the wavelength of the coherent light signal having the following behaviour:
λₜ(t)=−f(t)+C where C is a constant, between t₃ and t₄ where t₃ and t₄ ∈Δtᵢ.

22. The method according to claim 21, wherein all the sub sweeping intervals have a substantially equal sub sweeping duration Δt and λₜ₋₁(t)=−λₜ(t)+C where C is a constant for the whole duration of the sub sweeping interval.

23. The method according to claim 16, including:
dividing the sweeping in N, where N≥2, sub-sweeping intervals;
providing the (i−1)-th sub-sweeping interval having a duration Δtᵢ₋₁ with the wavelength of the coherent light signal having the following behaviour:
λₜ₋₁(t)=f(t) where f(t) is a monotone function between t₁ and t₂, where t₁ and t₂∈Δtᵢ₋₁; and
providing the i-th sub-sweeping interval having a duration Δtᵢ with the wavelength of the coherent light signal having the following behaviour:
λₜ(t)=f(t)+C where C is a constant, between t₃ and t₄ where t₃ and t₄ E Δtᵢ.

24. The method according to claim 23, wherein all the sub sweeping intervals have equal sub sweeping duration Δt and λₜ₋₁(t)=λₜ(t)+C where C is a constant for the whole duration of the sub-sweeping interval.

25. The method according to claim 21, wherein f(t) is a substantially linear function.

26. The method according to claim 16, including:
dividing the sweeping in N, where N≥2, sub-sweeping intervals all of identical sub-sweeping duration Δt and the wavelength of the coherent light signal is a substantially periodic function with period Δt or 2 Δt.

27. The method according to claim 16, including the step of dividing the sweeping in N sub-sweeping intervals, wherein 2≤N≤15.

28. The method according to claim 16, wherein:
the light source (101) has a spectral bandwidth narrower than 40 nm.

29. A Swept Source Optical Coherence Tomography system (SS-OCT), the SS-OCT system including:
a. a light source, tunable over a spectral band, that generates a coherent light signal;
b. an optical interferometer for dividing the coherent light signal into a reference arm leading to a reference reflector and a sample arm leading to a sample;
c. an optical element to selectively direct a sample light signal exiting the sample arm to a specific portion of the sample, so that, for each selection operated at the optical element, a different specific portion of the sample is illuminated;
d. an optical detector for detecting an interference signal generated by a combination of reference and sample returning signals from the reference arm and from the sample arm, reflected by the reference reflector and the sample, respectively;
e. a processing unit, said processing unit being programmed for, for the same selection in the optical element illuminating a specific portion of the sample:
i. defining a sweeping time interval ΔT;
ii. changing the coherent light signal leading to the sample light signal illuminating the specific portion of the sample from a minimum wavelength to a maximum wavelength and in the same sweeping modifying the wavelength of the coherent light signal so that it reaches the same value between the minimum wavelength to the maximum wavelength at least twice during the sweeping;
iii. elaborating the detected interference signal for obtaining an OCT image of the specific portion of the sample.

30. The SS-OCT system according to claim 29, wherein the light source is a tunable laser source including a liquid crystal tunable element.
